(19) United States
(12) Patent Application Publication
(10) Pub. No.: US 2026/0216277 A1
(43) Pub. Date: Jul. 30, 2026

(54) TOPICAL COMPOSITIONS AND METHODS FOR TREATING ALOPECIA

(71) Applicant: Yissum Research Development Company of the Hebrew University of Jerusalem Ltd., Jerusalem (IL)

(72) Inventors: Simon BENITA, Tel Aviv (IL); Taher NASSAR, Kfar Tur'an (IL)

(73) Assignee: Yissum Research Development Company of the Hebrew University of Jerusalem Ltd., Jerusalem (IL)

(21) Appl. No.: 19/145,576

(22) PCT Filed: Jan. 8, 2024
(86) PCT No.: PCT/IL2024/050021

§ 371 Date: Jul. 3, 2025

Related U.S. Application Data
(60) Provisional application No. 63/479,008, filed on Jan. 9, 2023.

(51) Int. Cl. A61K 38/13; A61K 9/107; A61K 31/40; A61K 31/445; A61P 17/14

(52) U.S. Cl. CPC A61K 38/13; A61K 9/1075; A61K 31/40; A61K 31/445; A61P 17/14

(57) ABSTRACT

The invention is in the field of therapeutic compositions and methods for the treatment of inflammatory conditions and disorders of the skin and hair, and alopecia in particular.

### TECHNOLOGICAL FIELD

The invention generally pertains to the field of therapeutic compositions and methods for the treatment of inflammatory conditions and disorders of the skin and hair, and alopecia in particular.

### BACKGROUND

Hair loss is a psychologically disturbing and mentally stressful, especially for women. There has been a plethora of studies in recent years on causes and therapeutic strategies for hair loss. The underlying causes of hair loss are diverse, among them hormonal imbalance (androgenic alopecia, AGA), chronic inflammatory condition associated with autoimmune diseases (alopecia areata, AA) and chemotherapy-induced alopecia (CIA). Both AGA and AA include inflammation involving the production of activated NKG2D⁺CD8⁺ cells that produce Th1 cytokine interferon γ. This chronic localized inflammation around the hair follicle leads to a local imbalance of immune tolerance and localized apoptosis, and ultimately to noticeable hair loss at that site.

The most common type of hair loss is AGA, characterized by progressive hair loss resulting from hair follicle miniaturization, the underlying causes of which are androgens levels and genetic variations in the androgen receptor gene. Males with AGA have about 50 times higher level of 5-α-reductase, the enzyme responsible for the conversion of testosterone into dihydrotestosterone (DHT) in hair scalp, than males without AGA.

Despite the existence of several oral or topical treatments for AGA, e.g., minoxidil for male and female hair loss, finasteride for males and cyclosporine A (CsA) [1-2], none of them proved to be restorative [3]. CsA is a known immunosuppressant used in the context of post-transplant organ rejection and certain autoimmune diseases. It is known to inhibit helper T-cells activation and suppress interferon γ production. Its potential applicability to AGA stemmed from observations of hypertrichosis in patients treated with oral CsA, which prompted researchers to suggest oral CsA for male pattern alopecia (MPA, a form of AGA) and AA. However, subsequent trials with either oral CsA alone or in conjunction with systemic steroids yielded questionable and often unpredictable outcomes—a success rate of 25-77% [4]. This is apart from the known risks of systemic CsA therapy, such as elevated blood pressure, changes in blood sugar, gastrointestinal problems, tremors, numbness, and others.

AA imposed a specific problem. Among several systemic and topical treatments suggested by experts for this condition [4], none were approved by the FDA until 2022. These included topical scalp treatments, such as a first-line treatment with a potent topical corticosteroid applied daily for at least 6 to 12 weeks for 3 to 6 months, and topical CsA for treating scalp and eyebrow AA, but not as a first-line treatment for beard AA. In June 2022, the FDA approved baricitinib, an immunomodulator and a Janus kinase (JAK) inhibitor, as a primary treatment for AA with indications of oral daily doses of 2 or 4 mg during the period of 36 weeks [5-6]. There are, however, some remaining questions as to effectiveness and safety of this treatment for AA, on individual and large scale.

In summary, the current situation is apparent insufficiency of effective and generally safe treatments for all forms of alopecia, and especially AA, which is even more exacerbated by the magnitude of the problem—the lifetime incidence of AA is approximately 2% worldwide in both sexes. Therefore, there is an unmet need to find an efficient and safe topical treatment that could prevent, reduce and recover hair loss in AGA, AA and other forms of alopecia.

### PUBLICATIONS

1. Gupta A K et al 2022. Comparison of oral minoxidil, finasteride, and dutasteride for treating androgenetic alopecia. J Dermatolog Treat 33:2946-2962.
2. Goldust M et al 2022. Does topical minoxidil in concentrations higher than 5% provide additional clinical benefit?Clin Exp Dermatol 47:1951-1955.
3. Santos Z et al 2015. Drug discovery for alopecia: Gone today, hair tomorrow. Expert Opin Drug Discov 10:269-292.
4. Meah N et al 2020. The Alopecia Areata Consensus of Experts (ACE) study: Results of an international expert opinion on treatments for alopecia areata. JAAD 83:123-130.
5. King B et al 2022. Two Phase 3 trials of baricitinib for alopecia areata. NEJM 386:1687-1699.
6. Lensing M and Jabbari A 2022. An overview of JAK/STAT pathways and JAK inhibition in alopecia areata. Front Immunol 13:955035.

### GENERAL DESCRIPTION

Topical drug delivery would be a preferred option for treating alopecia. In general, orally prescribed drugs may have safety considerations regarding related adverse effects, which is one of the reasons why only few oral drugs have been approved as systemic treatments for alopecia. Oral cyclosporine A (CsA), for example, has been related to a relatively high adverse effects profile, including among others nephrotoxicity, immune-suppression, hypertension, neuropathies, and a relatively high relapse rate.

The main objective in the context of skin delivery would be to assist dermal permeability of drugs in question. In this connection, CsA has very poor skin permeability, mainly due to its molecular weight and low water solubility. The skin and scalp penetration of CsA can be improved by incorporation of permeation enhancers such as ethanol, terpenes, others, and/or design of specific delivery systems such as nanoemulsion, liposomes and nano-capsules (NCs) incorporating the active, as has been reported for minoxidil and finasteride. Another advantage of such delivery systems is that they accumulate in the bulge region of hair follicle and serve as a drug reservoir.

Tempol (4-hydroxy-2,2,6,6-tetramethylpiperidine-1-oxyl) is a non-toxic synthetic antioxidant that is known to promote the metabolism of many reactive oxygen species (ROS) and reduce oxidative stress. It has high water solubility and low molecular weight, which allows it to permeate through biological membranes. It was further demonstrated to reduce inflammation, most likely by the reduction of leukocytes infiltration and activation of the Nrf2 signaling pathway. A sporadic small-scale study on whole brain radiotherapy suggested that topical application of Tempol to the scalp before radiation is relatively safe and well tolerated and can be protective against radiation-induced alopecia. However, subsequent attempts to use certain formulations of Tempol in this context were inconsistent and yielded no publishable results.

The incentive for the present technology was to design and develop a safe and effective topical delivery system with putatively effective actives for the treatment of AGA and AA, such as CsA and Tempol. As of now, AA is lacking effective topical treatment. CsA and Tempol are different by the physical and chemical properties, and the mechanism of action, and could be potentially complementary. Surprisingly, current studies have shown that in terms of efficacy, safety, skin penetration, and protective and restorative effects on AGA and AA, the CsA-Tempol combination is multipotent and synergistic, and thus can provide an effective solution not only to alopecia but also to other common skin conditions.

Ultimately, the anti-inflammatory capacities and topical applicability of formulations of the invention, as supported by the examples and discussion provided herein, make them especially attractive candidates for a wide range of additional common skin conditions involving topical inflammation, dyspigmentation, blemishes and scarring, certain examples are acne, atopic dermatitis, epidermolysis bullosa, hidradenitis supporative (HS), ichthyosis, pachyonychia congenita, pemphigus, psoriasis, Raynaud's phenomenon, rosacea, scleroderma, and vitiligo.

In the broadest sense, the invention can be articulated in terms of topical formulations comprising a matrix polymeric emulsifier composed or formed of a crosslinked copolymer of acrylic acid and an acrylate and a nanoemulsion comprising a lipophilic immunosuppressant and a cyclic nitroxide (spin label).

In some embodiments the topical formulation of the invention can be a gel formulation comprising a matrix polymeric emulsifier composed of a crosslinked copolymer of acrylic acid and an acrylate, e.g., a C₁₀-C₃₀ alkyl acrylate, and a nanoemulsion comprising a lipophilic immunosuppressant, and a cyclic nitroxide.

The term “gel formulation” encompasses herein aqueous, emulsified, and non-aqueous gel formulations. Typically, the gel formulation comprises an amount of an aqueous medium or water enabling stability of the nano emulsion. The gel formulations are typically formulated for topical use and may be characterized by a permeation profile of actives. The gel formulation typically comprises a matrix material, typically in the form of a polymeric emulsifier. The emulsifier may be a high molecular weight copolymer of acrylic acid and an acrylate such as a C₁₀-C₃₀ alkyl acrylate.

In some embodiments, crosslinking between the acrylic acid and acrylate, e.g., alkyl acrylate such as a C₁₀-C₃₀ alkyl acrylate, is achievable by the use of allyl pentaerythritol.

In some embodiments, the gel formulation comprises a matrix material in a form of a polymeric emulsifier selected from high molecular weight copolymer of acrylic acid and an acrylate such as a C₁₀-C₃₀ alkyl acrylate, wherein the two are crosslinked with allyl pentaerythritol.

In some embodiments, the gel formulation comprises a matrix material in a form of a polymeric emulsifier selected from high molecular weight copolymer of acrylic acid and an acrylate such as a C₁₀-C₃₀ alkyl acrylate, wherein the two are crosslinked with allyl pentaerythritol. The polymeric emulsifier is a material having CAS no. 138789-85-2.

In some embodiments, the matrix material is a Pemulen gel, which is a type of a pharmaceutical excipient with effective emulsification properties to form stable oil-in-water emulsions. Pemulen polymer excipients contain both hydrophilic and hydrophobic portions and are thus capable of creating a network around suspended oil droplets and providing exceptional emulsion stability, often without the need for additional surfactants. The term “Pemulen” refers herein to polymeric emulsifiers that are high molecular weight copolymers of acrylic acid and C₁₀-C₃₀ alkyl acrylate crosslinked with allyl pentaerythritol. The Pemulen refers to the polymeric emulsifier material having CAS no. 138789-85-2.

The copolymer of acrylic acid and the acrylate referred to as a C₁₀-C₃₀ alkyl acrylate, which is formed by crosslinking with allyl pentaerythritol, is a copolymer of different alkyl acrylate materials with acrylic acid. The “C₁₀-C₃₀ alkyl” is not means to encompass any one alkyl, rather a mixture of such defined alkyl acrylates that together with acrylic acid are crosslinked with allyl pentaerythritol to provide the copolymer.

In some embodiments, the gel formulations of the invention can further comprise at least one thickening agent to increase viscosity and topical applicability.

Types of applicable thickening materials that may be used according to some embodiments include:
Synthetic ingredients: polymers (polyacrylates and their derivatives carbopol, polycarbophil, etc.), poloxamers, polypropylene glycols, and waxes, wherein each material constitutes a separate and an independent embodiment of the invention;
Natural ingredients: polysaccharides (celluloses, hyaluronic acids, and chitosans, their derivatives, wherein each constitutes a separate and an independent embodiment of the invention);
Polyproteins: (natural, gelatins, collagen, and synthetic, poly-amino acids such as poly glutamic acid, wherein each constitutes a separate and an independent embodiment of the invention);
Silicones derivatives: as minerals clays such as magnesium aluminum silicate and polymer-based silicon such as elastomers wherein each constitutes a separate and an independent embodiment of the invention.

In some embodiments the thickening agent can be selected from polyacrylates and their derivatives, carbopol, polycarbophil, poloxamers, polypropylene glycols, waxes, polysaccharides, celluloses, hyaluronic acids, chitosans their derivatives, natural polyproteins, gelatins, collagen, poly-amino acids, mineral clays, magnesium aluminum silicate and polymer-based silicon.

In some embodiments the gel formulations of the invention can further comprise a nanoemulsion of an immunosuppressant, e.g., a lipophilic immunosuppressant, and a cyclic nitroxide. The term “immunosuppressant” encompasses herein any agent that any agent capable of reducing or suppressing the activity of at least one biomarker of immune system or a marker of inflammation in vivo or in vitro.

In some embodiments the chosen immunosuppressant can be a steroid agent, a cell proliferation inhibitor, an antibody, an immunophilin-based drug, mycophenolate, a tumor necrosis factor (TNF-α) inhibitor, and others. Non-limiting examples of immunosuppressants include azathioprine (Imuran), Cyclosporine A (CsA), Mercaptopurine (Purinethol, 6-MP), rapamycin, fujimycin and methotrexate.

In some embodiments, the immunosuppressant can be selected from azathioprine (Imuran), Cyclosporine A (CsA), Mercaptopurine (Purinethol, 6-MP), rapamycin, fujimycin and methotrexate.

In some embodiments, the immunosuppressant can be CsA.

In some embodiments, the nanoemulsion and the matrix material can be mixed into a stable gel as defined herein.

The cyclic nitroxide used in formulations of the invention is a spin label cyclic compound having a ring nitrogen atom that is bonded to an oxygen atom with an unpaired electron. Typically, the cyclic nitroxide is of the structure (I) as defined herein. Alternatively, the cyclic nitroxide is a cyclic compound which may or may not have an endocyclic double bond and/or one or more substituting moiety, and a cyclic oxygen atom with an unpaired electron.

### In Some Embodiments, the Gel Formulation can Comprise

(1) a matrix of a crosslinked copolymer of acrylic acid and a C₁₀-C₃₀ alkyl acrylate and
(2) a nanoemulsion comprising an immunosuppressant (which may be lipophilic), and a cyclic nitroxide, wherein the cyclic nitroxide is a compound of a formula I:

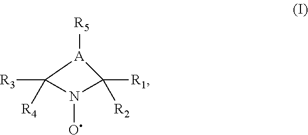

wherein
A represents a carbon atom or a carbon chain comprising up to three carbon atoms (e.g., to provide a ring structure of 4, 5 or 6 atoms; in case of A being a 3-carbon atom chain-to provide a 6-membered heterocyclic ring structure, such as piperidinyl or TEMPOL; in case of A being a 2-carbon atom chain- to provide a 5-membered ring heterocyclic structure such as pyrrolidinyl or PROXYL, optionally comprising one or more double bonds, i.e., pyrroline a structure), wherein at least one (or only one) of the carbon atoms may be substituted with an oxygen atom or an oxygen containing group (oxazolinyl or DOXYL), and/or wherein one or more of the carbon atoms is substituted with one or two bromine atoms,
each of R₁, R₂, R₃ and R₄, independently, is selected from H and C₁-C₅ alkyl; or
each of R₁ and R₂ together with the carbon atom to which they are attached form a 3- to 7-membered cyclic ring, and/or
each of R₃ and R₄ together with the carbon atom to which they are attached form a 3- to 7-membered cyclic ring,
R₅ represents a group selected from aldehydes, ketones, carboxylic acids, carbonyl groups, —O—, —S—, —OH, —SH, —COOH, —COONH₂, —CN, and primary-(e.g., —NH₂), secondary—(e.g., —NH—R), tertiary-(e.g., —NR′R″) or quaternary-amines (e.g., a charged amine), and wherein
O^{·} represents an oxygen radical.

Each of R, R′ and R″, independently, used in reference to the amine groups may be a C₁-C₅ alkyl or any other carbon group.

Group A may also or alternatively be selected from carbon groups that form endocyclic double bonds with a neighboring carbon atom. For example, group A may be of the form ═CR5, wherein the carbon atom designated ═C forms a double bond with a vicinal carbon atom.

In some embodiments, the cyclic nitroxide compound can be a 5-membered heterocyclic ring structure.

In some embodiments, the cyclic nitroxide compound can be a 6-membered heterocyclic ring structure.

In some embodiments, in the cyclic nitroxide compound of formula (I), each of R₁, R₂, R₃ and R₄ is H or a C₁-C₅ alkyl.

In some embodiments, in the cyclic nitroxide compound of formula (I), each of R₁, R₂, R₃ and R₄ is different from H.

In some embodiments, in the cyclic nitroxide compound of formula (I), each of R₁, R₂, R₃ and R₄ is a C₁-C₅ alkyl selected from methyl, ethyl, propyl, isopropyl, butyl, and pentyl.

In some embodiments, in the cyclic nitroxide compound of formula (I), each of R₁, R₂, R₃ and R₄ is a different C₁-C₅ alkyl, namely having a different number of carbon atoms or a different structure.

In some embodiments, in the cyclic nitroxide compound of formula (I), each of R₁, R₂, R₃ and R₄ is a linear C₁-C₅ alkyl.

In some embodiments, in the cyclic nitroxide compound of formula (I), each of R₁, R₂, R₃ and R₄ is a methyl group.

In some embodiments, the cyclic nitroxide compound of formula (I) can be a compound of formula (II):

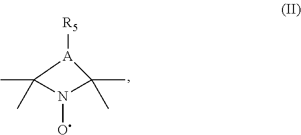

wherein each of A and R₅ is as defined above.

In some embodiments, in the cyclic nitroxide compound of formula (I) or formula (II), A is a carbon group comprising one, two or three carbon atoms, at least one of said carbon atoms being bonded to an oxygen containing group. The oxygen containing group may be a hydroxyl group, or an ether group or wherein the oxygen containing group is an oxygen containing group selected amongst such groups defining R₅. In other words, the group A-R₅ may be a group selected from A=O, A-OH, A-COOH and A-COONH₂ wherein A is optionally a carbon atom or a carbon group, as defined, wherein the carbon atom may be as SP3 or an SP2 carbon atom.

When referring to a “carbon group”, it should be clear that the group comprises one or more carbon atoms and one or more other atoms, e.g., H or heteroatoms, such that the carbon group is arranged to provide a linear, branched or interrupted carbon chain. Typically, the carbon group contains 1 to 3 carbon atoms, wherein each carbon atom is further bonded to hydrogen atoms or other atoms, as specified, to provide complete and correct atom valences. In some embodiments, A is a group having a carbon skeleton selected from —C—, —C—C—, and —C—C—C—, and a suitable number of H atoms, wherein one of the hydrogen atoms may be substituted with an oxygen containing group, as defined, and all other substitutions are hydrogen atoms. In some embodiments, A is a group having a carbon skeleton selected from —C—, —C—C—, and —C—C—C—, wherein one of the hydrogen atoms is substituted with an oxygen containing group, as defined, and all other substitutions are hydrogen atoms, or wherein one or two of the carbon atoms are substituted with one or two Br atoms and all other substitutions are hydrogen atoms.

In some embodiments, A is a group having or forming at least one endocyclic double bond.

In some embodiments, A is a group selected from —CH—, —CH—CH₂—, —CH—CH₂—CH₂— and —CH₂—CH—CH₂—, and wherein the group A-R₅ is selected from —CHR₅—, —CHR₅—CH₂—CHR₅—CH₂—CH₂— and —CH₂—CHR₅—CH₂—.

In some embodiments, the group A-R₅ is —CHR₅—, —CHR₅—CH₂—CH₂—, or —CH₂—CHR₅—CH₂—, wherein R₅ is an oxygen containing group, as defined.

In some embodiments, the group A-R₅ is —CH(OH)—, —CH(OH)—CH₂—CH₂—, or —CH₂—CH(OH)—CH₂—.

In some embodiments, the group A-R₅ is —CH₂—CH(OH)—CH₂—.

In some embodiments, the cyclic nitroxide compound is a 5-membered heterocyclic ring structure, wherein the group A-R₅ is —CH₂—CH(OH)—CH₂—.

In some embodiments, the cyclic nitroxide compound is a 6-membered heterocyclic ring structure, wherein the group A-R₅ is —CH₂—CH(OH)—CH₂—.

In some embodiments, the cyclic nitroxide may be 4-hydroxy-2,2,6,6-tetramethylpiperidin-1-oxyl, Tempol:

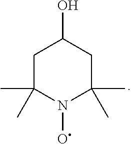

In some embodiments the cyclic nitroxide compound can be of the formula:

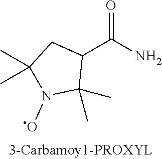

In some embodiments the cyclic nitroxide compound can be of the formula:

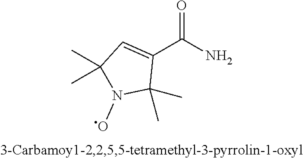

In some embodiments, the cyclic nitroxide can be selected from 3-Carbamoyl-PROXYL, 4-hydroxy-2,2,6,6-tetramethylpiperidin-1-oxyl, and 3-Carbamoyl-2,2,5,5-tetramethyl-3-pyrrolin-1-oxyl.

In some embodiments, the nanoemulsion can comprise a lipophilic immune-suppressant such as CsA, and a cyclic nitroxide selected from 3-Carbamoyl-PROXYL, 4-hydroxy-2,2,6,6-tetramethylpiperidin-1-oxyl, and 3-Carbamoyl-2,2,5,5-tetramethyl-3-pyrrolin-1-oxyl.

In some embodiments, the gel formulation can comprise at least one of:
(1) a matrix of a crosslinked copolymer of acrylic acid and a C₁₀-C₃₀ alkyl acrylate and
(2) a nanoemulsion comprising a lipophilic immunosuppressant such as CsA, and a cyclic nitroxide selected from 3-Carbamoyl-PROXYL, 4-hydroxy-2,2,6,6-tetramethylpiperidin-1-oxyl, and 3-Carbamoyl-2,2,5,5-tetramethyl-3-pyrrolin-1-oxyl.

In some embodiments, the formulation can comprise CsA and Tempol.

In some embodiments, the formulation can comprise CsA at a concentration in the range of about 0.01% to about 0.5% (w/w), or more specifically at concentrations in the range of about 0.01-0.05%, 0.05-0.1%, 0.1-0.2%, 0.2-0.3%, 0.3-0.4%, 0.4-0.5% (w/w), or concentrations in the range of up to 0.01%, 0.05%, 0.1%, 02%, 0.3%, 0.4%, 0.5% (w/w).

In some embodiments, the formulation can comprise Tempol at a concentration in the range of about 0.1% to about 5% (w/w), or more specifically at concentrations in the range of about 0.1-0.5%, 0.5-1%, 1-1.5%, 1.5-2%, 2-2.5%, 2.5-3%, 3-3.5%, 3.5-4%, 4-4.5% or 4.5-5% (w/w), or concentrations in the range of up to 0.1%, 0.5%, 1%, 1.5%, 2%, 2.5%, 3%, 3.5%, 4%, 4.5% and 5% (w/w).

In some embodiments, the formulation can comprise CsA at the concentration of about 0.1% and Tempol at the concentration of about 0.5% (w/w).

In some embodiments, the gel formulations can further comprise one or more additional actives or inert additives. Depending on the type of formulation desired, and a variety of other factors, hair growth preparations may include various ingredients which may be mixed and dissolved, as known in the pharmaceutical and/or cosmetic fields. Generally speaking, formulations of the invention may comprise diluents, buffers, flavors, binders, surfactants, thickeners, lubricants, preservatives, pH adjusters, fungicides, antioxidants, emulsifiers, stabilizers, spices and colorants.

One of the characteristics features of the present formulations is revealed in the particle size of the entrapped nanoemulsion.

In some embodiments, the nanoemulsion can have a particle size in the range of about 200 nm to about 300 nm, or more specifically particle size can range between 200 and 210, 210 and 220, 220 and 230, 230 and 240, 240 and 250, 250 and 260, 260 and 270, 270 and 280, 280 and 290 or between 290 and 300 nm.

In some embodiments, the particle size can range between 100 and 110, 110 and 120, 120 and 130, 130 and 140, 140 and 150, 150 and 160, 160 and 170, 170 and 180, 180 and 190, 190 and 200, 200 and 210, 210 and 220, 220 and 230, 230 and 240, 240 and 250, 250 and 260, 260 and 270, 270 and 280, 280 and 290, 290 and 300, 300 and 310, 310 and 320, 320 and 330, 330 and 340, 340 and 350, 350 and 360, 360 and 370, 370 and 380, 380 and 390, 390 and 400, 400 and 410, 410 and 420, 420 and 430, 430 and 440, 440 and 450, 450 and 460, 460 and 470, 470 and 480, 480 and 490 or between 490 and 500 nm.

In some embodiments, the nanoemulsion can be entrapped in the crosslinked copolymer of acrylic acid and C₁₀-C₃₀ alkyl acrylate matrix to the extent of 60% to 100%, or more specifically the nanoemulsion can be entrapped up to 60%, 65%, 70%, 75%, 80%, 85%, 90%, 95% or 100% in the crosslinked copolymer of acrylic acid and C₁₀-C₃₀ alkyl acrylate matrix, or entrapped in a range of 60 and 65%, 70 and 75%, 80 and 85%, 90 and 95% or 95 and 100% in the matrix.

In some embodiments, the gel formulation can remain stable at 37° C. over a period of at least 3, 6, 9, 12, 15, 18, 21, 24, 27, 30, 33, 36, 39, 42, 45, 48, 51 weeks or more, or over the period of at least 1, 2, 3, 4, 5, 6, 7, 8, 9, 10, 11, 12 months or more, or over the period of at least 1, 2, 3, 4, 5, 6 years or more. Stability can be measured and verified in terms of preservation of the concentration of actives over time or physical properties of the formulation, such as viscosity. Both measurements have been presently exemplified.

For example, the viscosity of the gel formulation can remain relatively constant or stable at 37° C. to the extent of ±20% over the period of at least 1, 2, 3, 4, 5, 6, 7, 8, 9, 10, 11, 12 months.

In some embodiments, the gel formulation can have preferential distribution into deeper dermal layers, such as the epidermis and/or dermis. The term “preferential” implies herein an increased distribution, amount or concentration of at least one active in the epidermis and/or dermis relative to the distribution, amount or concentration thereof in other tissues and specifically, the stratum corneum.

In some embodiments, the gel formulation can have an increased permeation into the epidermis and dermis layers of the skin relative to the permeation of the formulation into the stratum corneum layer. The term “permeation” implies herein an increase amount or concentration of at least one active in the epidermis and/or dermis relative to the amount or concentration thereof in the stratum corneum.

The degree of increase in the amount or concentration the active in the epidermis and/or dermis can be in the range of up to 10%, 20%, 30%, 40%, 50%, 60%, 70%, 80%, 90%, 100%, 150%, 200%, 250%, 300%, 350%, 400%, 450%, 500% or more relative to the amount or concentration thereof in the stratum corneum, or up to 2-, 3—, 4-, 5-, 6-, 7-, 8-, 9-, 10-, 15-, 20-, 25-, 30-fold and more relative to the amount or concentration thereof in the stratum corneum. Such measurements have been presently exemplified.

It is another aspect the invention to provide clinical and cosmetic methods and uses of the gel formulations, and their permutations according to the present definitions, for promoting hair growth and/or reconstitution of dermal histomorphology on the human skin or scalp. These effects can be evaluated by known qualitative and quantitative methods, for example visual or quantitative evaluation of hair density and histological evaluation of skin morphology. Such measurements have been presently exemplified.

The term “hair growth” encompasses herein maintenance, induction, stimulation, promotion and regeneration of hair development in a human or an animal subject. Additionally, it encompasses growth of defective hair, prolongation of the anagen stage in the pilar cycle and conversion of vellus hair to terminal hair.

In some embodiments, and especially when using the formulation comprising CsA and Tempol, the effect of CsA and Tempol on the treating or promoting hair growth and/or reconstitution of dermal histomorphology on the human skin or scalp can be synergistic.

The term “synergistic” implies that the effect or the gel formulation with the combined actives (CsA and Tempol) exceeds the effect of a gel formulation with a single active (CsA or Tempol) by at least 2-, 3-, 4-, 5-, 6-, 7-, 8-, 9-, 10-fold.

In yet another aspect, the invention provides methods and uses of the above gel formulations in treating alopecia in its various clinical presentations.

The term “alopecia” encompasses herein all types of defective hair growth and partial or entire hair loss, including but not limited to a male hormonal alopecia, androgenic alopecia (AGA), toxic alopecia, alopecia areata (AA), telogen alopecia, alopecia due to endocrine abnormalities, metabolic disorders and nutritional disorders, pharmaceutical alopecia, mechanical alopecia, alopecia due to skin diseases, scarring alopecia, congenital alopecia, and trichotillomania.

In some embodiments, the alopecia is androgenic alopecia (AGA) or alopecia areata (AA).

One aspect to be considered in the context of clinical methods and uses is the effective amount, concentration or dose actives applied to treated subject, implied by the term “therapeutically effective” amount, concentration, or dose. This term typically refers to the amounts, concentrations, or doses or actives that have been related to noticeable or measurable therapeutic effects by recognizable clinical standards, such as reduction of clinical symptoms of the disease or condition, reduction of one or more recognized biomarkers thereof, patient's reporting on improvement of clinical symptoms, etc.

In some embodiments, the improvement or reduction of clinical symptoms or biomarkers as above can involve daily, weekly or monthly topical administering of therapeutically effective amounts of the gel formulations of the invention.

In some embodiments, the improvement or reduction of clinical symptoms or biomarkers as above can involve topical administering of therapeutically effective amounts of the formulations one or more times a day.

In yet another aspect, the invention provides use of the present gel formulations, and their permutations in the manufacture of a topical medicament for promoting hair growth and/or reconstitution of dermal histomorphology on the human skin or scalp.

In some embodiments the invention provides use of the present gel formulations in the manufacture of a topical medicament for various manifestations of alopecia.

Topical formulations of the invention may be developed into a plurality of forms, including liquid or semi-liquid preparations such as lotions, emulsions, creams, ointments, liniments, sprays, aerosols, oils, pastes, gels, tonics, solutions or suspensions, and be adapted for scalp and/or skin applications.

More broadly, because of their anti-inflammatory effects and topical applicability, it is most likely that the formulations of the invention can be used in a wide range of additional common skin conditions, and especially the group of conditions commonly referred to as inflammatory, immune and/or auto-immune skin conditions. The most common examples are widely occurring topical skin inflammation, dyspigmentation, blemishes and scarring. Additional clinically relevant examples are acne, atopic dermatitis, epidermolysis bullosa, hidradenitis supporative (HS), ichthyosis, pachyonychia congenita, pemphigus, psoriasis, Raynaud's phenomenon, rosacea, scleroderma, and vitiligo. Studies on potential applicability of the formulations to such conditions are currently ongoing.

The term “about” generally implies herein at least ±5%, ±10%, ±15%, ±20%, ±25% deviation from the respective values.

### The Invention Further Provides:

A topical formulation comprising a matrix polymeric emulsifier composed of a crosslinked copolymer of acrylic acid and an acrylate and a nanoemulsion comprising an immunosuppressant and a cyclic nitroxide.

In any configuration of a formulation disclosed herein, the formulation may be a gel formulation selected from aqueous gel formulations, emulsified gel formulations, and non-aqueous gel formulations.

In any configuration of a formulation disclosed herein, the matrix polymeric emulsifier is a high molecular weight copolymer of acrylic acid and an acrylate.

In any configuration of a formulation disclosed herein, the acrylate may be a C₁₀-C₃₀ alkyl acrylate.

In any configuration of a formulation disclosed herein, the matrix polymeric emulsifier may be selected from high molecular weight copolymer of acrylic acid and an acrylate, wherein the acrylic acid and the acrylate are crosslinked with allyl pentaerythritol.

In any configuration of a formulation disclosed herein, the formulation comprises at least one thickening agent.

In any configuration of a formulation disclosed herein, the thickening agent is selected amongst polyacrylates and their derivatives, carbopol, polycarbophil, poloxamers, polypropylene glycols, waxes, polysaccharides, celluloses, hyaluronic acids, chitosans their derivatives, natural polyproteins, gelatins, collagen, poly-amino acids, mineral clays, magnesium aluminum silicate and polymer-based silicon.

In any configuration of a formulation disclosed herein, the nanoemulsion and the matrix polymeric emulsifier are provided as a stable gel.

In any configuration of a formulation disclosed herein, the immunosuppressant is selected amongst such agents that reduce or suppress the activity of an in vivo immune system, optionally selected from a steroid agent, a cell proliferation inhibitor, an antibody, an immunophilin-based drug, mycophenolate, and a tumor necrosis factor (TNF-α) inhibitor.

In any configuration of a formulation disclosed herein, the immunosuppressant is selected from azathioprine (Imuran), Cyclosporine A (CsA), Mercaptopurine (Purinethol, 6-MP), rapamycin, fujimycin and methotrexate.

In any configuration of a formulation disclosed herein, the immunosuppressant is CsA.

In any configuration of a formulation disclosed herein, the formulation comprising:
a matrix of a crosslinked copolymer of acrylic acid and a C₁₀-C₃₀ alkyl acrylate; and
a nanoemulsion comprising a lipophilic immunosuppressant, and a cyclic nitroxide, wherein the cyclic nitroxide is a compound of a formula I:

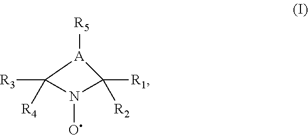

wherein
A represents a carbon atom or a carbon chain comprising up to three carbon atoms, wherein one of the carbon atoms is substituted with an oxygen atom or an oxygen containing group, or wherein one or more of the carbon atoms is substituted with one or two bromine atoms,
each of R₁, R₂, R₃ and R₄, independently, is selected from H and C₁-C₅ alkyl; or
each of R₁ and R₂ together with the carbon atom to which they are attached form a 3- to 7-membered cyclic ring, and/or each of R₃ and R₄ together with the carbon atom to which they are attached form a 3- to 7-membered cyclic ring,
R₅ represents a group selected from aldehydes, ketones, carboxylic acids, carbonyl groups, —O—, —S—, —OH, —SH, —COOH, —COONH₂, —CN, and primary-, secondary-, tertiary- or quaternary-amines, and wherein
O— represents an oxygen radical.

In any configuration of a formulation disclosed herein, the cyclic nitroxide compound is a 5-membered heterocyclic ring structure.

In any configuration of a formulation disclosed herein, the cyclic nitroxide compound is a 6-membered heterocyclic ring structure.

In any configuration of a formulation disclosed herein, the cyclic nitroxide compound is a 5-membered heterocyclic ring structure comprising an endocyclic double bond.

In any configuration of a formulation disclosed herein, the cyclic nitroxide compound of formula (I), each of R₁, R₂, R₃ and R₄, independently, is C₁-C₅ alkyl.

In any configuration of a formulation disclosed herein, the cyclic nitroxide compound of formula (I), each of R₁, R₂, R₃ and R₄, independently, is selected from methyl, ethyl, propyl, isopropyl, butyl, and pentyl.

In any configuration of a formulation disclosed herein, the cyclic nitroxide compound of formula (I), each of R₁, R₂, R₃ and R₄ is a methyl group.

In any configuration of a formulation disclosed herein, the cyclic nitroxide compound of formula (I) is a compound of formula (II):

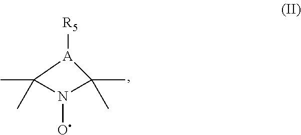

wherein each of A and R₅ is as defined herein.

In any configuration of a formulation disclosed herein, the cyclic nitroxide compound of formula (I) or formula (II), A is a carbon group comprising one, two or three carbon atoms, at least one of said carbon atoms being bonded to an oxygen containing group.

In any configuration of a formulation disclosed herein, the oxygen containing group is a hydroxyl group, or an ether group or wherein the oxygen containing group is an oxygen containing group selected amongst from R₅.

In any configuration of a formulation disclosed herein, the group A-R₅ is a group selected from A=O, A-OH, A-COOH and A-COONH₂.

In any configuration of a formulation disclosed herein, the A-R₅ is —CHR₅—, —CHR₅—CH₂—, —CHR₅—CH₂—CH₂—, or —CH₂—CHR₅—CH₂—.

In any configuration of a formulation disclosed herein, the group A-R₅ is —CHR₅—, —CHR₅—CH₂—CH₂—, or —CH₂—CHR₅—CH₂—, wherein R₅ is an oxygen containing group.

In any configuration of a formulation disclosed herein, the group A-R₅ is —CH(OH)—, —CH(OH)—CH₂—CH₂—, or —CH₂—CH(OH)—CH₂—.

In any configuration of a formulation disclosed herein, the group A-R₅ is —CH₂—CH(OH)—CH₂—.

In any configuration of a formulation disclosed herein, the cyclic nitroxide compound is a 5-membered heterocyclic ring structure, wherein the group A-R₅ is —CH₂—CH(OH)—.

In any configuration of a formulation disclosed herein, the cyclic nitroxide compound is a 6-membered heterocyclic ring structure, wherein the group A-R₅ is —CH₂—CH(OH)—CH₂—.

In any configuration of a formulation disclosed herein, the cyclic nitroxide is Tempol:

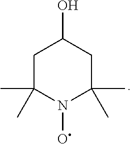

In any configuration of a formulation disclosed herein, the cyclic nitroxide compound is of the formula:

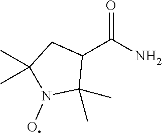

In any configuration of a formulation disclosed herein, the cyclic nitroxide compound is of the formula:

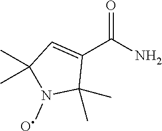

In any configuration of a formulation disclosed herein, the cyclic nitroxide is selected from 3-Carbamoyl-PROXYL, 4-hydroxy-2,2,6,6-tetramethylpiperidin-1-oxyl, and 3-Carbamoyl-2,2,5,5-tetramethyl-3-pyrrolin-1-oxyl.

In any configuration of a formulation disclosed herein, the nanoemulsion comprises CsA, and a cyclic nitroxide selected from 3-Carbamoyl-PROXYL, 4-hydroxy-2,2,6,6-tetramethylpiperidin-1-oxyl, and 3-Carbamoyl-2,2,5,5-tetramethyl-3-pyrrolin-1-oxyl.

In any configuration of a formulation disclosed herein, the formulation comprises at least one of:
a matrix of a crosslinked copolymer of acrylic acid and a C₁₀-C₃₀ alkyl acrylate, and
a nanoemulsion comprising CsA, and a cyclic nitroxide selected from 3-Carbamoyl-PROXYL, 4-hydroxy-2,2,6,6-tetramethylpiperidin-1-oxyl, and 3-Carbamoyl-2,2,5,5-tetramethyl-3-pyrrolin-1-oxyl.

In any configuration of a formulation disclosed herein, the nanoemulsion has a particle size in the range of about 200 nm to about 300 nm.

In any configuration of a formulation disclosed herein, the nanoemulsion is entrapped in the crosslinked copolymer of acrylic acid and C₁₀-C₃₀ alkyl acrylate matrix to the extent of 60% to 100%.

In any configuration of a formulation disclosed herein, the formulation is stable at 37° C. over a period of at least 3 months.

In any configuration of a formulation disclosed herein, the formulation has a preferential distribution into deeper dermal layers.

In any configuration of a formulation disclosed herein, the formulation having an increased permeation into the epidermis and dermis layers of the skin relative to the permeation of the formulation into the stratum corneum layer.

In any configuration of a formulation disclosed herein, the formulation comprising CsA and Tempol.

In any configuration of a formulation disclosed herein, the CsA is at a concentration in the range of about 0.01% to about 0.5% and the Tempol is at a concentration in the range of about 0.1% to about 5% (w/w).

In any configuration of a formulation disclosed herein, the concentration of CsA is about 0.1% and the concentration of Tempol is about 0.5% (w/w).

In any configuration of a formulation disclosed herein, the formulation is for use in treating or promoting hair growth and/or reconstitution of dermal histomorphology on the human skin or scalp.

In any configuration of a formulation disclosed herein, the formulation is for use in treating or promoting hair growth and/or reconstitution of dermal histomorphology on the human skin or scalp, wherein the effect of CsA and Tempol on the treating or promoting hair growth and/or reconstitution of dermal histomorphology on the human skin or scalp is synergistic.

Also provided is use of a formulation as disclosed herein in a method of preventing or treating alopecia.

In any configuration of a formulation disclosed herein, the alopecia is selected from male hormonal alopecia, androgenic alopecia (AGA), toxic alopecia, alopecia areata (AA), telogen alopecia, alopecia due to endocrine abnormalities, metabolic disorders and nutritional disorders, pharmaceutical alopecia, mechanical alopecia, alopecia due to skin diseases, scarring alopecia, congenital alopecia, and trichotillomania.

In any configuration of a formulation disclosed herein, the alopecia is androgenic alopecia (AGA) or alopecia areata (AA).

Also provided is a method for treating male or female alopecia, in a subject in need thereof, the method comprising topical administering to the subject a therapeutically effective amount of the formulation as disclosed herein.

A method of treating or promoting hair growth and/or reconstitution of dermal histomorphology on the human skin or scalp, the method comprising topical administering to a subject a therapeutically effective amount of the formulation as disclosed herein.

A method of treating inflammatory, immune and/or auto-immune skin conditions in a subject, the method comprising topical administering to the subject a therapeutically effective amount of the formulation as disclosed herein.

In any configuration of a formulation disclosed herein, the formulation is for use in treating human inflammatory, immune and/or auto-immune skin conditions.

In any configuration of a formulation disclosed herein, the use is in the manufacture of a topical medicament for treating human inflammatory, immune and/or auto-immune skin conditions.

In any configuration of a formulation disclosed herein, the use is in the manufacture of a topical medicament for treating or promoting hair growth and/or reconstitution of dermal histomorphology on the human skin or scalp.

Also provided is a formulation comprising a nanoemulsion of CsA and a cyclic nitroxide selected from 3-Carbamoyl-PROXYL, 4-hydroxy-2,2,6,6-tetramethylpiperidin-1-oxyl, and 3-Carbamoyl-2,2,5,5-tetramethyl-3-pyrrolin-1-oxyl; and a copolymer of acrylic acid and a C₁₀-C₃₀ alkyl acrylate, wherein the formulation is configured for application onto a skin region of a subject.

In any configuration of a formulation disclosed herein, the nanoemulsion is entrapped in the copolymer, and wherein the CsA is at a concentration in the range of about 0.01% to 0.5% and the cyclic nitroxide is at a concentration in the range of about 0.1% to 5% (w/w).

In any configuration of a formulation disclosed herein, the formulation comprising 4-hydroxy-2,2,6,6-tetramethylpiperidin-1-oxyl for use in a method of preventing or treating alopecia.

### BRIEF DESCRIPTION OF THE DRAWINGS

Specific embodiments will now be described by way of non-limiting examples with reference to the following drawings.

FIG. 1 illustrates certain morphological features of the present nanoemulsions, i.e., the relatively uniform nanometric particle size and high encapsulation efficiency (EE) of CsA, as revealed by TEM images of the blank nanoemulsions (A1/A2) and 0.5% CsA nanoemulsions (B1/B2), TEM and cryo-TEM respectively.

FIG. 2 illustrates certain morphological features of the present gel formulations, i.e., the micrometric matrix structure with improved stability and complete incorporation of the CsA loaded nanoemulsion, as revealed by SEM images of the blank gels (A1/A2) and 0.5% CsA gels (B1/B2), in x10 and x20 magnifications respectively.

FIG. 3 illustrates additional physicochemical properties of the present gel formulations, i.e., non-Newtonian pseudoplastic character and prolonged stability, as revealed by viscosity measurements of the blank (▴), 0.1% CsA (□), 0.5% Tempol (●) and 0.1% CsA-0.5% Tempol (∘) gels at 37° C. All gel formulations had relatively constant viscosity at 37° C. for at least 6 weeks, the CsA-Tempol gel had the highest viscosity (about 17.7 Pa·S).

FIG. 4 illustrates the feature of improved stability at 37° C. during the period of 12 weeks (about 3 months), reproducing the same phenomenon of constant stability of all gel formulations—the blank (▴), 0.1% CsA (□), 0.5% Tempol (● dashed line) and 0.1% CsA-0.5% Tempol (● solid line) gels, with the highest more advantageous viscosity for the CsA-Tempol gel.

FIG. 5 illustrates the cytotoxic effect of the present delivery systems, as revealed by viability studies in HaCaT cells treated (24 h) with different concentrations of blank gel (Pemulen gel), CsA gel and free CsA and testing by MTT assay (mean±SD. N=3). CsA was nontoxic at concentrations below 5 μg/mL, both in gel and free drug forms, blank Pemulen gel was nontoxic at higher concentration (2 mg/mL).

FIGS. 6A-6B illustrate the feature of improved permeation into the deeper skin layer, as revealed by comparative analysis of CsA amounts in the stratum corneum (SC), epidermis and dermis of fresh (living) and frozen human skin ex vivo treated with CsA in oil, and CsA nanoemulsion and gel preparations (mean±SD, N=9 mice×3 donors, by two-way ANOVA Dunnett multiple comparison, *p<0.05, **p<0.01, ***p<0.001, ****p<0.0001). The behavior of the CsA gel in the frozen and living tissues was similar, with preferential permeation into the deeper layers of the epidermis and dermis.

FIGS. 7A-7D illustrate the feature of improved permeation under more controlled conditions, using frozen human skin ex vivo treated CsA in oil, and CsA nanoemulsion and gel preparations and measurements of CsA amounts in SC, epidermis and dermis at 2, 4, 6 and 24 h timepoints (mean±SD, N=9 mice×3 donors, by two-way ANOVA Dunnett multiple comparison, *p<0.05, **p<0.01, ***p<0.001, ****p<0.0001). Again, the CsA gel had a strong preferential penetration into the epidermis and dermis, while the CsA nanoemulsion had an inclination to remain in the SC.

FIGS. 8A-8I illustrate the immunosuppressive effect of CsA-Tempol gel on topical inflammation in vivo, as revealed by histological analysis of skin sections obtained from DNFB induced animal model, including: sham mice (non-induced) (A), mice exposed to DNFB induced ear inflammation (no treatment) (B), mice treated with 0.1%, 0.2%, 0.5% CsA gels (C-E), mice treated with 0.1%, 0.2%, 0.5% CsA-5% Tempol gels (F—H) and mice with the 5% Tempol gel only (I). All treatment groups showed very good recovery from topical inflammation, which by the histology was essentially normal (see sham 8A).

FIGS. 9-11 illustrate the immunosuppressive effect of the formulations by the profiles of three main proinflammatory cytokines, TNF-α (FIG. 9), IL-6 (FIG. 10) and INF-γ (FIG. 11) at the DNFB-induced site (mean±SD, N=5 mice by ANOVA Dunnett normality test, *p<0.05, **p<0.002, *** p<0.001). Both CsA and Tempol had strong reducing effects on cytokines levels. The 0.2% CsA-5% Tempol and 0.5% CsA-5% Tempol gel formulations had significant effects on the reversal of inflammation by the levels at least TNF-α and IL-6.

FIG. 12 illustrates the restorative effect of the formulations on hair growth in AGA animal model, including: sham, alopecia model (non-treated) groups, and groups treated with blank gel (placebo), 0.5% CsA nanoemulsion, 0.5% CsA in castor oil, 0.5% CsA gel, 5% Tempol gel and 0.5% CsA-5% Tempol gel. Quantitative analysis of hair restoration (pixel density by area under curve, AUC %) on day 14 (mean±SD, N=8 mice by ANOVA Dunnett normality test vs. sham, *p<0.05, **p<0.002, ***p<0.001) suggested superior performance of the CsA-Tempol gel compared to other formulations, which surpassed the hair density in sham.

FIGS. 13A-13H illustrate the restorative effect on hair growth by histological sections (H & E staining) obtained from the same experiment, showing yet again a superior effect of the gel formulations, CsA-Tempol (13h) and CsA gel (13c), in terms of thickness of dermis and number of hair follicles and overall recovery of the primary dermal structure, which was similar to sham (13a). The other treatments were similar to the alopecia model (13b), with a thin dermis and a pronounced deficiency of hair follicles.

FIGS. 14-15 illustrate the same effect as revealed by another experiment in the AGA animal model, using only gel formulations with lower concentrations of actives, including: sham, alopecia model (non-treated) groups and groups treated with 0.1% CsA gel, 0.5% Tempol gel or 0.1% CsA-0.5% Tempol gel formulations. FIG. 14 shows animals on days 2, 10, 12 and 14 after treatment. Both sham (14a) and CsA-Tempol groups (14e) showed significant hair regrowth on day 12 and full hair restoration on day 14 compared to the partial effects of CsA (13c) and Tempol (13d) gels. FIG. 15 illustrates hair regrowth by quantitative analysis of the same animals (hair regrowth by AUC %), showing yet again a superior effect of the 0.1% CsA-0.5% Tempol gel formulation compared to the other gels (mean±SD on day 14, N=5 mice by ANOVA Dunnett normality test vs. sham, **p<0.002).

### DETAILED DESCRIPTION OF EMBODIMENTS

Topical nanoemulsions based drug delivery systems, in general, attracted much attention owing to their ability to minimize adverse effects and enhance skin permeation. The inventors of present technology developed novel composite nanoemulsion gel formulations of CsA and Tempol that proved to be exceptionally effective for topical applications onto the skin. The proof of concept was provided by the current example of incorporation of a CsA nanoemulsion in castor oil and Tempol into Pemulen gel. Current studies included detailed characterization of this formulation and its derivatives, including physicochemical properties, encapsulation, recovery rate and stability studies, and studies of skin penetration and clinical effects in human keratinocytes in vitro and samples of human skin ex vivo, and further in an established murine model of AGA using 5α-androstane-3β, 17β-diol (DHT), where it proved to be effective even with low concentrations of CsA and Tempol, the lowest reported so far in animal models of AGA and AA.

More specifically, a CsA nanoemulsion gel was produced by dispersing a CsA-loaded nanoemulsion in a pseudoplastic crosslinked copolymer of acrylic acid and alkyl acrylate co-monomer aqueous gel (Pemulen). The composite CsA-Tempol gel formulation was obtained by dissolving Tempol in the CsA nanoemulsion gel. The CsA nanoemulsions had a relatively uniform particle size (150-200 nm, PDI<0.20), the particle size and zeta potential increased with increased CsA loading (^{~}25-^{~}35 mV). The CsA nanoemulsions have a high CsA recovery (about 90%) and encapsulation efficiency (EE, about 85%). The gel formulations had an increased particle size and a lower zeta potential (240-260 nm, ~45-^{~}55 mV), showing that the gel improved electrostatic stability and minimized droplet coalescence. Stability studies of the gels showed that the CsA and Tempol actives remain stable for at least 12-weeks period (3 months) at 37° C., current studies are testing 6 months stability. Rheological analysis showed that the gels exhibited Newtonian pseudoplastic properties, with increased viscosity in drug-loaded gel formulations. (9.2-17.7 Pa·S).

Overall, the incorporation of CsA nanoemulsions into Pemulen crossed network has led to significant advantages of improved physical and chemical stability, reduced tendency to coalescence of oil droplet and reduced risk of CsA leakage. More generally, hydrogels are the preferred type of topical formulations, constituting over 80% of the entire market size.

Preliminary cytotoxicity studies human keratinocytes cell line showed that the CsA gel formulation is safe and nontoxic at the CsA concentrations of <5 μg/mL. Pemulen gel was also nontoxic up to the concentration of 2 mg/mL (relevant for topical applications). Permeation studies in the human skin model ex vivo showed that upon topical application the gel formulations had superior drug permeation kinetics, with a preferential distribution of the drug (CsA) into the epidermis and dermis, the latter is the target tissue where the hair follicles are located. The maximal amount of CsA in the dermis (about 6.5 μg/g tissue) was after 24 h with the gel treatment, in which it outperformed the nanoemulsion and oil formulations.

The main problems with topical use of nanoemulsions are: (1) their fluidity and unsuitability for localized application, and (2) their tendency to coalesce and increase droplet size that reduces their permeation into the deeper skin tissues, specifically the dermis where the drug is most needed for alopecia. The gel, owing to the good bio-adhesion and other properties, prevented the coalescence of oil droplets, reduced drug leakage, and further provided an improved permeation of the drug into the deeper layers of the skin.

Further studies in the established animal model of inflammation using repeated topical application of DNFB to mouse ear showed that while the histology of inflamed ears was characterized by significant epidermal thickness and infiltration of immune cells compared to sham (non-induced) controls, the histology of animals treated with CsA or/and Tempol gel formulations was similar to sham. These observations were further supported on the level of specific cytokines, showing that the treatments with the CsA and Tempol gels significantly reduced the levels of TNF-α and IL-6, two important proinflammatory cytokines, and potentially IFN-γ. Overall these studies supported the notion that gel formulations of CsA and Tempol, alone and in combination, can act as effective immunosuppressors.

Finally, studies in an established model of AGA showed that gel formulations, CsA Tempol and the CsA-Tempol combination, were more effective in inducing hair regrowth compared to the oil control and nanoemulsion formulations. The 0.1% CsA+0.5% Tempol combination was the most effective, showing the fastest densest hair regrowth that was even superior to sham. These findings were further supported by histological analyses of the mouse skin sections, showing that the treatments with CsA and CsA-Tempol gels had histological manifestations that were similar to sham in terms of the thickness of dermis and the density hair follicles. Furthermore, the gel with the CsA-Tempol combination was more successful with lower concentrations of actives, 0.1% CsA and 0.5% Tempol compared to 0.5% CsA alone, thus providing efficacy and safety at the same time.

Altogether, these findings provide proof of concept for topical applicability of a new gel formulation combining two known actives, CsA and Tempol, for effective treatment of alopecia. Clinical efficacy and safety of this formulation was demonstrated so far in an established animal model of AGA, where it achieved significant synergistic therapeutic effects with reduced concentrations of both actives, which are unlikely to be systemically hazardous or have significant side effects. Importantly, the CsA and Tempol gel formulation proved to be effective on several levels: (1) in restoring normal hair growth, (2) restoring the original dermal morphology, and (3) reducing local inflammation.

Regarding AA, which is another common clinical manifestation of alopecia, AA is an autoimmune disease characterized by non-scarring hair loss, sometimes involving the entire body. Hair loss is associated with infiltration of self-reactive T cells into hair follicles, leading to inflammation and the formation of reactive oxygen species. Based on present findings in the model of AGA, there is a good prospect that the CsA and Tempol gel formulation is likely to be effective in for AA, and other rarer forms of alopecia.

Some embodiments of the invention will be now described by way of examples with reference to respective figures.

### Example 1: Physicochemical Properties of Gel Formulations

### 1.1 Material and Methods

### 1.1.1 Materials

CsA was obtained from Teva (Israel); Tempol (4-hydroxy-2,2,6,6-tetramethylpiperidine-1-oxyl), Polysorbate 80, (Tween® 80), sorbitan monooleate (Span® 80), Cremophor® EL, 3-(4,5 dimethyl-2-thiazolyl)-2,5-diphenyl-2H-tetrazolium bromide (MTT), 1-Fluoro-2,4-dinitrobenzene (DNFB) and 5α-androstane-30,170-diol (DHT) from Sigma-Aldrich (Israel); [³H]-CsA from ARC American Radiolabeled Chemicals, Inc. (USA); Ultima-Gold® liquid scintillation cocktail and Solvable® from Perkin-Elmer (USA); Macrogol 15 hydroxystearate (Solutol™ HS15) from BASF (Germany); Dimethyl sulfoxide (DMSO) from Fluka (Switzerland); Glycerol tributyrate from Alfa Aesar (UK); Castor oil from Tamar Laboratory Supplies Ltd (Israel); Glycerol anhydrous, hydrogen peroxide 30% and all organic solvents HPLC grade from J. T. Baker (Holland); Tissue culture media from Biological Industries Ltd (Israel); HaCaT cells (human keratinocytes cell line) from PromoCell (Germany); Pemulen™ TR-2, a high molecular weight, crosslinked copolymer of acrylic acid and a hydrophobic C₁₀₋₃₀ alkyl acrylate co-monomer, from BF Goodridge (USA); and Lipoid E80 from Lipoid GmbH, Ludwigshafen, Germany.

### 1.1.2 Preparation and Characterization of CsA Nanoemulsions

The CsA nanoemulsion was prepared by solvent displacement method. In brief, cyclosporine (CsA), Tween 80 and castor oil were dissolved in acetone (9 mL). Lipoid E80 was prepared in ethanol (1 mL) and added to the acetone solution (10 mL organic phase), and stirred, 1600 rpm, 30 min. The organic phase was added dropwise into DDW (20 mL) containing 0.1% w/v of Solutol SH 15, stirred, 1000 rpm, 15 min. Acetone was evaporated by Rota evaporator. The gel formulations were prepared on the basis of three source nanoemulsions with various CsA concentrations (0.05%, 0.1%, 0.2% and 0.5% w/w) and other ingredients as detailed in Table 1 below.

TABLE 1
Composition of the CsA nanoemulsions

| Ingredients | Amounts | |
|---|---|---|
| Cyclosporine | 10, 20, 40 or 100 | mg |
| Castor oil | 250 | mg |
| Solutol SH 15 | 50 | mg |
| Tween 80 | 100 | mg |
| Lipoid E80 | 50 | mg |
| Acetone | 9 | mL |
| Ethanol | 1 | mL |
| Water | 20 | mL |

### 1.1.3 Preparation of CsA-Tempol Gel

Tempol was dissolved in the CsA nanoemulsions to produce CsA-Tempol nanoemulsions. In brief, CsA nanoemulsions (20 mL with 0.05%, 0.1%, 0.2%, 0.5% CsA w/w) were mixed with various concentrations of Tempol (0.05%, 0.1%, 0.5%, 2%, 5% w/w), 1500 rpm, 3 min, as detailed in Table 2 below. CsA gel was produced by dispersion of the CsA nanoemulsions in the Pemulen polymer. Pemulen (0.25% w/w) was added to CsA nanoemulsions or CsA-Tempol nanoemulsions to produce respective CsA gel and CsA-Tempol gel formulations. In the latter, Pemulen (50 mg) were dispersed in CsA-Tempol nanoemulsion (20 mL) while stirring, 1 M NaOH (18 μL) were added to produce gel formulations. Placebo gels were prepared by dispersing Pemulen in blank nanoemulsion, glycerol (5% w/w) was added to prevent skin dryness.

TABLE 2
Composition of the CsA Tempol gels

| Tempol | Amounts | |
|---|---|---|
| 0.05% | 10 | mg |
| 0.1% | 20 | mg |
| 0.5% | 100 | mg |
| 2% | 400 | mg |
| 5% | 1000 | mg |

### 1.1.4 Physicochemical Characterization of the Formulations

Mean particle size, polydispersity index (PDI) and zeta potential of the formulations, nanoemulsions (10 μL) and gels (20 mg) diluted in DDW (2 mL), were evaluated using Zeta Sizer Nano (ZSP, 4 mW 632.8 nm “red” laser) at 25° C., multimodal narrow modes (high resolution), display range 0.6-6000 nm (average of 3 measurements per sample, 14 runs per measure).

### 1.1.5 CsA/Tempol Determination

Quantification of CsA and Tempol was performed by HPLC (Thermo Scientific, USA equipped with Xterra C8 column). The preparations (20 μL) were dissolved in acetonitrile (980 μL), the mobile phase was an isocratic mixture of acetonitrile (70%) and water (30%) at a constant flow rate of 0.5 mL·min⁻¹. UV detection was performed at 210 nm for CsA and 234 nm for Tempol, column temperature was maintained at 60° C., sample injection volume at 10 μL with partial mode.

### 1.1.6 Encapsulation Efficiency and Recovery Rate

Drug recovery rate (%) was calculated as in Eq 1 below. Encapsulation efficiency (EE %) was calculated as in Eq 2.

$$recovery \% = [ \frac{drug weight in emulsion}{initial drug weight} ] \times 100 \qquad ( Eq . 1 )$$

$$EE \% = [ \frac{drug weight in emulsion - free drug weight}{initial drug weight} ] \times 100 \qquad ( Eq . 2 )$$

### 1.1.7 Transmission Electron Microscopy (TEM)

CsA and blank nanoemulsions were analyzed by TEM (Jem-1400 Plus Microscope, JEOL USA) with phosphotungstic acid staining as contrasting agent. Briefly, samples (5 μL) placed on formvar/carbon coated copper grids (200 mesh, EMS), mixed with PTA (2%, 5 μL), excess volume was blotted, and grids were air-dried.

### 1.1.8 Cryo-Transmission Electron Microscopy (Cryo-TEM)

Cryo-TEM enables direct imaging without contrasting agents. Samples were prepared by applying 2-3 μL nanoemulsion to a glow discharge treated TEM grid (300 mesh Cu Lacey substrate, Ted Pella, Ltd, Redding, USA). Excess liquid was blotted, the specimen was vitrified in liquid nitrogen (Vitrobot Mark IV, FEI), and examined by FEI Tecnai 12 G2 TWIN TEM operated at 120 kV.

### 1.1.9 Scanning Electron Microscopy (SEM)

CsA and blank gels topography were examined by scanning electron microscopy (SEM). Briefly, gel formulations (10 mg) were smeared on glass (1×1 cm²) and air-dried overnight at RT. Gels were sputter-coated with iridium/gold mixture prior to the analysis. Images were taken at various positions in a low and high.

### 1.1.10 Rheological Studies of the Gels

Rheological behavior was examined by rheometer using flat-plate sensor (101 PP-25, Anton Paar, Austria). Flow curves were analyzed by ramping the shear rate from 0 to 1000 s⁻¹ in 120 sec, shear stress was recorded throughout the experiment.

### 1.2 Results and Discussion

### 1.2.1 Physicochemical Characterization of CsA Nanoemulsions

Physicochemical properties of the nanoemulsions are summarized in Table 3 below. In summary, the CsA nanoemulsions had a relatively narrow size distribution (PDI<0.20), mean particle diameter in the range of about 150-190 nm, CsA loading slightly increased particle size, with more than 90% CsA recovery rate and 85% EE.

TABLE 3
Physicochemical properties of the CsA loaded nanoemulsions

| | Drug | Mean | | Zeta | CsA |
|---|---|---|---|---|---|
| | amount | diameter | | Poten.^{b} | recovery |
| Formulation | % w/v | nm | PDI^{a} | mV | % |
| Blank emulsion | — | 154 ± 5 | 0.08 | −26 ± 2 | — |
| CsA emulsion | 0.1 | 170 ± 7 | 0.11 | −27 ± 2 | 96 ± 2 |
| CsA emulsion | 0.2 | 175 ± 4 | 0.09 | −26 ± 2 | 95 ± 3 |
| CsA emulsion | 0.5 | 189 ± 4 | 0.120 | −32 ± 3 | 92 ± 2 |

^{a}PDI = poly dispersity index,
^{b}Zeta Potential

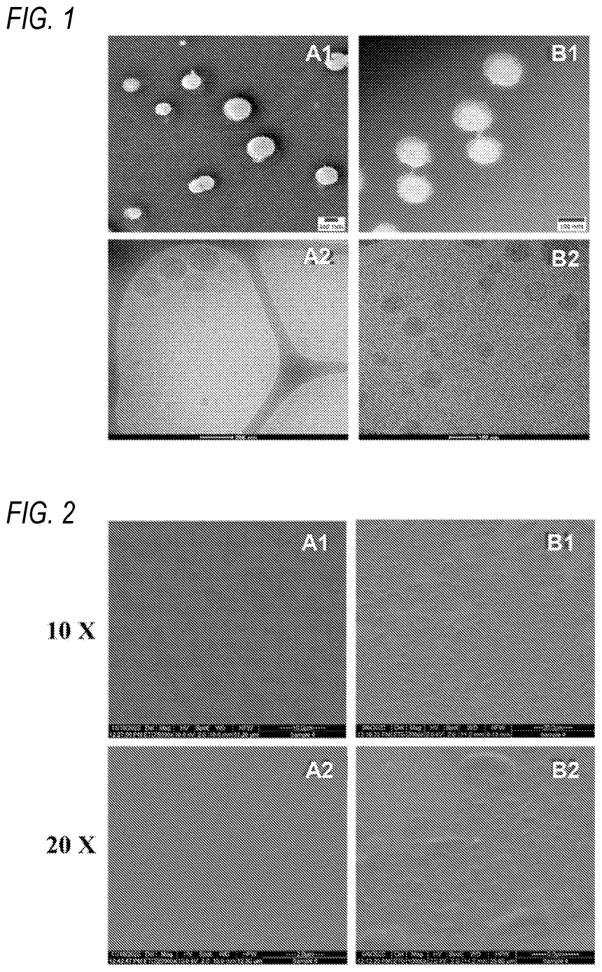

TEM and cryo-TEM imaging analysis showed that both blank and CsA (0.5%) nanoemulsions are highly monodispersed and spherical in shape (see FIG. 1). In addition, CsA was completely dissolved in the delivery system, with no traces of CsA particles outside the spheres (no undissolved CsA).

### 1.2.2 Physicochemical Characterization of the Gel Formulations

SEM analysis of the blank and 0.5% CsA gel formulations showed that the nanoemulsion droplets were completely immersed in the gel matrix (FIG. 2). Physicochemical properties of the gel formulations with various concentrations of CsA and Tempol, loading of actives, particle size and zeta potential, are summarized in Table 4. Chemical stability of the two actives, CsA and Tempol, in selected gel formulations is provided in Table 5.

TABLE 4
Physicochemical properties of the gel formulations

| | CsA | Tempol | Mean | | Zeta | |
|---|---|---|---|---|---|---|
| Gel | conc. | conc. | diameter | | Poten.^{b} | |
| formulation | % w/v | % w/v | nm | PDI^{a} | mV | pH |
| Blank gel | — | — | 239 ± 2 | 0.377 | −52 ± 2 | 5.4 ± 0.3 |
| 0.5% CsA | 0.512 ± 0.01 | — | 258 ± 4 | 0.325 | −51 ± 3 | 5.7 ± 0.2 |
| 5% Tempol | — | 5.01 ± 0.03 | — | — | — | 5.5 ± 0.2 |
| 0.5% CsA | 0.511 ± 0.01 | 5.03 ± 0.05 | 242 ± 5 | 0.350 | −55 ± 2 | 5.3 ± 0.3 |
| 5% Tempol | | | | | | |
| 0.05% CsA | 0.052 ± 0.05 | — | 248 ± 5 | 0.388 | −58 ± 4 | 5.5 ± 0.5 |
| 0.05% Tempol | — | 0.049 ± 0.05 | — | — | — | 5.4 ± 0.2 |
| 0.05% CsA | 0.051 ± 0.04 | 0.052 ± 0.08 | 255 ± 5 | 0.350 | −47 ± 4 | 5.5 ± 0.2 |
| 0.05% Tempol | | | | | | |
| 0.1% CsA | 0.102 ± 0.05 | — | 245 ± 7 | 0.330 | −55 ± 3 | 5.9 ± 0.2 |
| 0.5% Tempol | — | 0.495 ± 0.06 | — | — | — | 5.4 ± 0.3 |
| 0.05% CsA | 0.105 ± 0.07 | 0.505 ± 0.06 | 255 ± 7 | 0.380 | −45 ± 3 | 5.2 ± 0.2 |
| 0.05% Tempol | | | | | | |

^{a}PDI = poly dispersity index,
^{b}Zeta Potential

TABLE 5
Chemical stability of the CsA and Tempol in selected gel formulations

| | CsA | Tempol | 0.1% CsA- | CsA | CsA |
|---|---|---|---|---|---|
| | 0.1% gel | 0.5% gel | 0.5% Tempol | 0.5% gel | 0.1% gel |
| Concentration | 100% | 100% | CsA 100% | Tempol 100% | 100% |
| (% w/v) | (0.102 ± 0.05) | (0.495 ± 0.06) | (0.105 ± 0.07) | (0.505 ± 0.06) | (0.512 ± 0.01) |
| Weeks 1 | 99.4 ± 0.4 | 99.5 ± 0.2 | 99.5 ± 0.4 | 99.7 ± 0.2 | 99.5 ± 0.3 |
| 2 | 99.5 ± 0.3 | 99.2 ± 0.5 | 99.5 ± 0.3 | 99.4 ± 0.3 | 99.5 ± 0.4 |
| 3 | 99.4 ± 0.3 | 98.6 ± 1 | 98.2 ± 1 | 98.7 ± 1 | 99.1 ± 0.5 |
| 4 | 98.7 ± 1 | 98.2 ± 1 | 97.5 ± 2 | 98.8 ± 1 | 98.5 ± 1 |
| 5 | 98.5 ± 2 | 97.5 ± 2 | 96.1 ± 4 | 97.5 ± 2 | 98.4 ± 1.1 |
| 6 | 98.6 ± 1 | 97.4 ± 1 | 96.3 ± 3 | 97.4 ± 2 | 98.2 ± 1.2 |
| 7 | 97.5 ± 2 | 97.1 ± 1 | 96.0 ± 2 | 96.5 ± 2 | 97.2 ± 2 |
| 8 | 97.6 ± 2 | 97.4 ± 2 | 95.5 ± 3 | 95.4 ± 3 | 97.5 ± 3 |
| 9 | 96.8 ± 2 | 97.0 ± 2 | 95.2 ± 4 | 95.2 ± 2 | 97.2 ± 2 |
| 10 | 96.5 ± 3 | 96.8 ± 2 | 95.2 ± 3 | 94.5 ± 2 | 96 ± 3 |
| 11 | 96.2 ± 2 | 96.4 ± 2 | 94.5 ± 4 | 94.2 ± 2 | 95.5 ± 4 |
| 12 | 96.3 ± 3 | 96.5 ± 3 | 94.0 ± 5 | 93.5 ± 2 | 95 ± 4 |

Values are mean ± SD, N = 3

The results show that all gel formulations were characterized by a relatively high loading capacity of actives, relatively uniform particle size in the range of 240-260 nm and zeta potential of about (−45)-(−55) mV. HPLC analysis of the two actives in selected the gel formulations showed that CsA and Tempol preserved chemical stability for a period of at least 6 weeks. In summary, the incorporation of CsA nanoemulsions into the Pemulen crossed network has led to significant advantages of improved physical and chemical stability, and upon equilibrium, to a lesser risk of CsA leakage from the oil droplets.

### 1.2.3 Rheological Behavior of the Gel Formulations

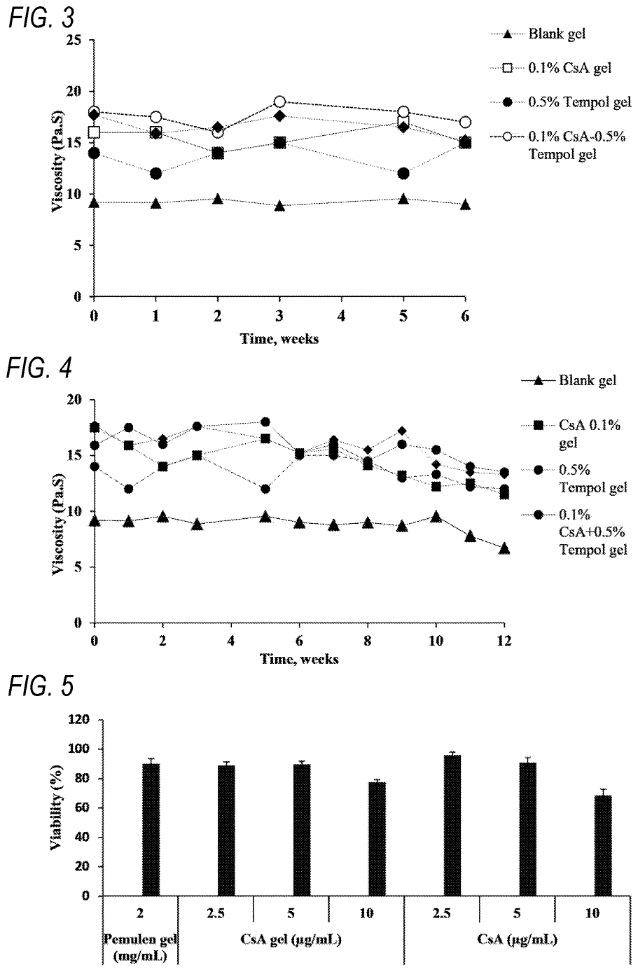

Rheological analysis showed that all gel formulations have non-Newtonian pseudoplastic character and a relatively constant viscosity at 37° C. for an extended period of time, i.e., at least 6 weeks (FIG. 3) and at least 12 weeks (FIG. 4). The 0.1% CsA-0.5% Tempol gels had the highest viscosity (about 17.7 Pa·S) compared to blank gel (about 9.2 Pa·S). A higher viscosity is more advantageous for topical applications.

### Example 2: Safety and Skin Permeation of the Gel Formulations

### 2.1 Material and Methods

### 2.1.1 In Vitro Cytotoxicity Analysis

HaCaT cells (human keratinocytes) were seeded in 96-well plates (10,000 cells/well) in DMEM with 10% FCS, streptomycin (0.1 mg/mL) and penicillin (100 units/mL) at 37° C. overnight. Medium was replaced with fresh medium (100 μL) with increasing concentrations of tested formulations. Cell viability was determined after 24 h by MTT assay according to the manufacturer's instructions, with measurements of OD at 570 nm and at 690 nm.

### 2.1.2 Ex Vivo Skin Penetration Analysis

Human skin samples were obtained from healthy adults after elective cosmetic surgery (abdominoplasty). Skin was freed from underlying fat and subcutaneous tissues, cut to pieces (2×2 cm), thinned to a thickness (0.75-1.0 mm) and stored at −80° C. For the experiment, thawed skin samples were mounted on Franz diffusion cells (Permagear Inc., USA), with diffusion area (1 cm²) and receptor compartment (8 mL) using receptor fluid (10% ethanol in PBS). The system was kept at 37° C., the skin surface temperature preserved at 32±1° C. Test samples (25 μL) containing 125 μg CsA (non-radiolabeled) and 125 μg [³H]-labelled CsA (1 milliCurie/mL) were applied on the skin. Skin samples were dismounted at different time intervals (2, 4, 6, 24 h) and washed with the receptor fluid (1 mL). Different skin layers were obtained in the following manner: (1 stratum corneum (SC) layers were removed by skin sampling discs (CuDERM Corp, USA), pooled and dissolved in DMSO; (2) epidermis layers were scraped off by scalpel; (3) dermis layers were cut into 1-2 mm pieces; the separated layers were chemically dissolved with Solvable and H₂O₂; (4) receptor fluids were also collected. Radioactivity measured by a scintillation counter (Tri-CARB 2900TR, Perkin Elmer, USA).

### 2.2 Results and Discussion

### 2.2.1 Lack of Cytotoxicity

Cytotoxic effect of various concentrations of CsA (2.5, 5, 10 μg/mL), in the form of gel and as free drug were tested compared to blank Pemulen gel (2 mg/mL). Overall, CsA was found nontoxic at concentrations below 5 μg/mL, both in gel and as a free drug (FIG. 5). Pemulen gel was also nontoxic at the concentration of 2 mg/mL, which is the concentration used in the topical applications.

### 2.2.2 Skin Permeation of CsA in Human Ex Vivo Model

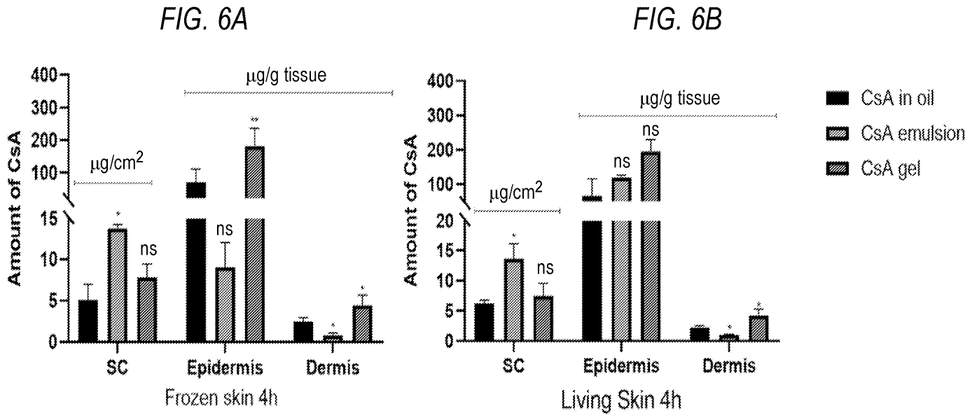

CsA skin permeation was studied upon topical application of the CsA oil, nanoemulsion and gel formulations on fresh (living) and frozen human skin tissues ex vivo. A preliminary study suggested that frozen and fresh tissues are essentially similar in terms CsA permeation profiles, both showing that the CsA gel provides preferential permeation of CsA into the deeper layer skin tissues i.e., the epidermis and dermis (FIGS. 6A-6B).

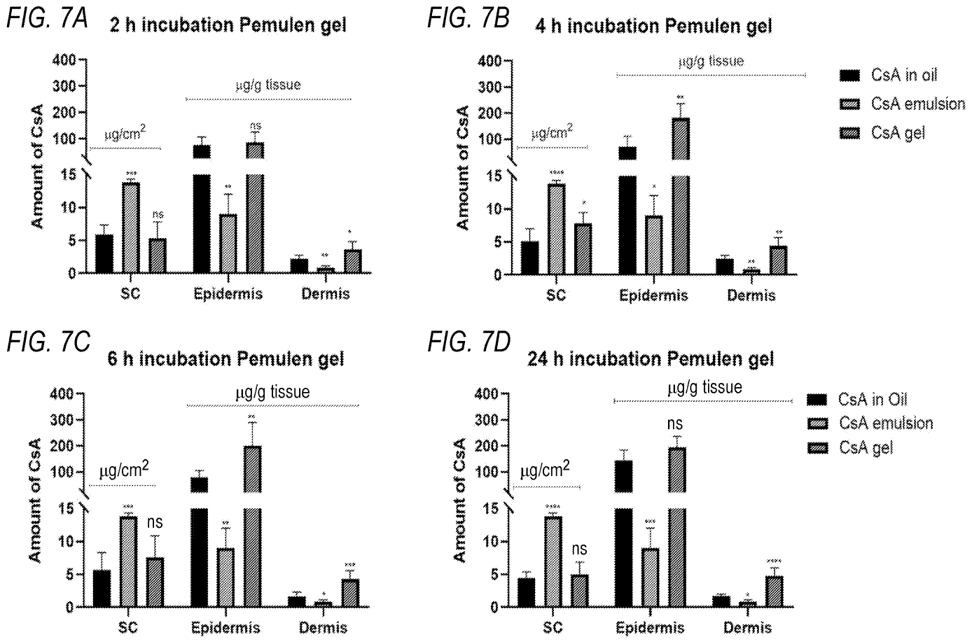

In a more detailed study, frozen human skin tissues as above were exposed to topical application of CsA oil, nanoemulsion and gel formulations, and CsA permeation into the SC, epidermis and dermis layers was monitored at 2, 4, 6 and 24 h timepoints. Essentially, the results supported the previous findings that the CsA gel provided preferential CsA permeation into the deeper skin layers, the epidermis and dermis. The CsA nanoemulsion, in contrast, was predominantly distributed in the SC (FIGS. 7A-7D).

The feature of improved permeation of active into the deeper skin layers, which is characteristic of the gel formulations but not nanoemulsions and free frug forms, is further goes together with the general benefits of gels for localized topical applications. Altogether, these studies suggest that the incorporation of CsA nanoemulsions into the Pemulen gel polymer matrix network provides a highly effective, stable and safe CsA formulation that is particularly advantageous for topical applications.

### Example 3: Immunosuppressive Effect of the CsA-Tempol in Gel Formulations

### 3.1 Material and Methods

### 3.1.1 In Vivo Inflammation Model

Inflammation studies used an established mouse model of topical inflammation, i.e., mouse inflamed ear following repeated exposure to DNFB. Briefly, 0.2% DNFB (50 μL in acetone and olive oil, ratio 4:1, respectively) was applied onto the shaved abdomen of mice (8-weeks old male C57BL/6 mice) for three successive days. Subsequently, the backside of the mice ears was brushed with 0.2% DNFB (10 μL) every alternate day, followed by topical application of 30 mg Tempol gel and CsA, and CsA-Tempol gel formulations with various concentrations of CsA. The experimental groups included: sham mice (A), inflammation model (B) (no treatment), mice treated with CsA gels (0.1%, 0.2% and 0.5% CsA) (C)-(E), mice treated with CsA-Tempol gels 0.1%, 0.2% and 0.5% CsA and 5% Tempol) (F)—(H) and mice treated with 5% Tempol gel (I). All mice were sacrificed on day 14 and skin tissues were analyzed by H & E staining. A schematic representation of the experimental procedure is shown below.

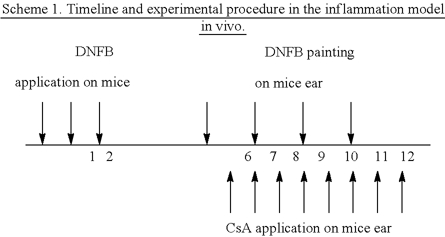

H & E staining used a standard protocol.

### 3.1.3 Biochemical Analysis

The levels of proinflammatory cytokines (TNF-α, IL-6 and IFN-γ) in the ear tissue were evaluated using ELISA. Briefly, 20-30 mg samples were homogenized (Bertin Technologies, USA) in PBS (1 mL) containing protease inhibitors (EZBlock, USA), 8 cycles of 30 sec at 4500 rpm with 40 sec breaks between cycles. The levels of TNF-α, IFN-γ and IL-6 in the lysates were determined according to the manufacturer's instructions (DuoSet ELISA, USA).

### 3.2 Results and Discussion

### 3.2.1 Histological Analysis of Topical Inflammation In Vivo

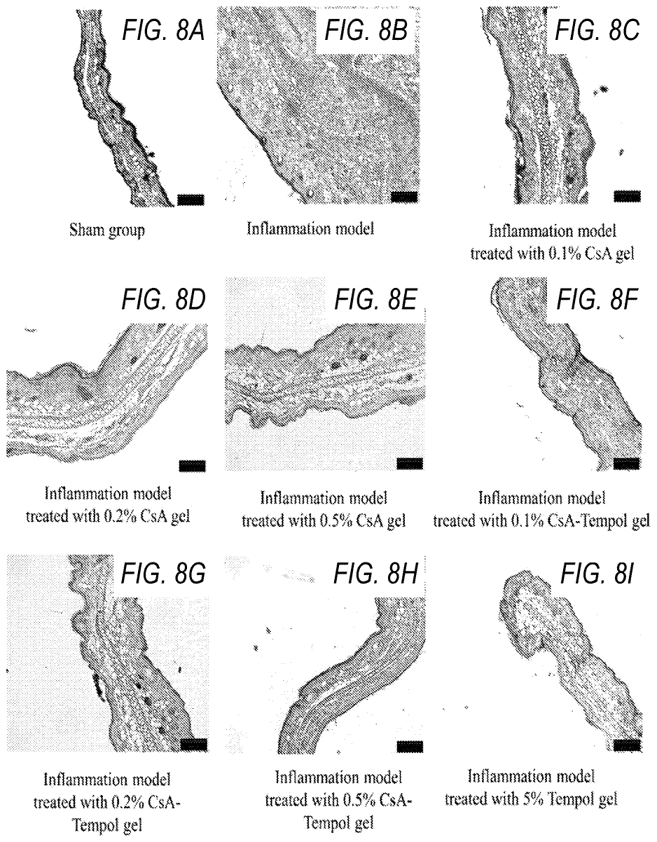

Histological results showed that all treatment groups, CsA and CsA-Tempol gels with various CsA concentrations and Tempol gel, prevented or significantly attenuated signs of inflammation, i.e., epidermal thickness and infiltration of immune cells into the skin tissue (FIGS. 8A-8I). The histological presentations of the treated groups were essentially similar to sham, and significantly different from the inflammation model (no treatment group), showing significant increase in epidermal thickness and massive infiltration of immune cells. These results suggest that both CsA and Tempol in gel formulations are effective suppressors of topical inflammation.

### 3.2.2 Analysis of Main Proinflammatory Cytokines

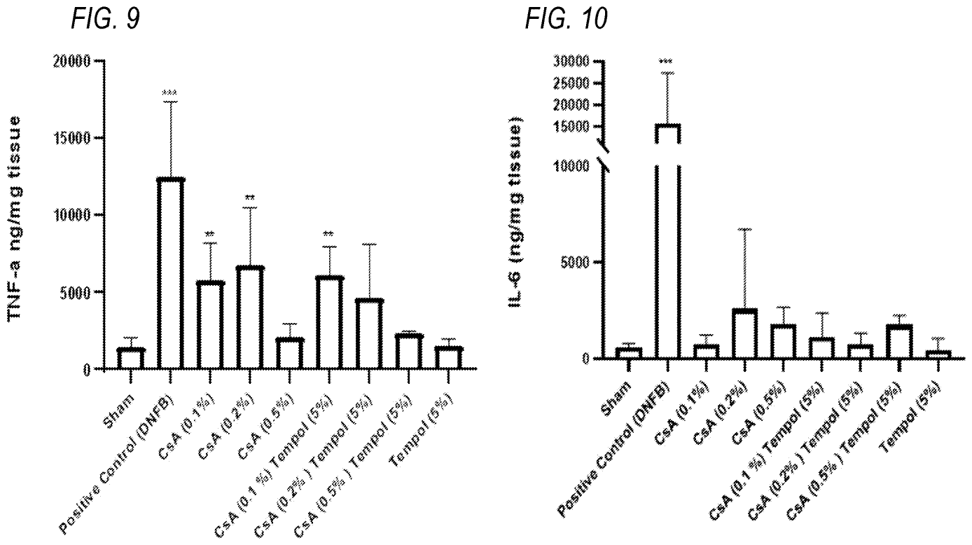

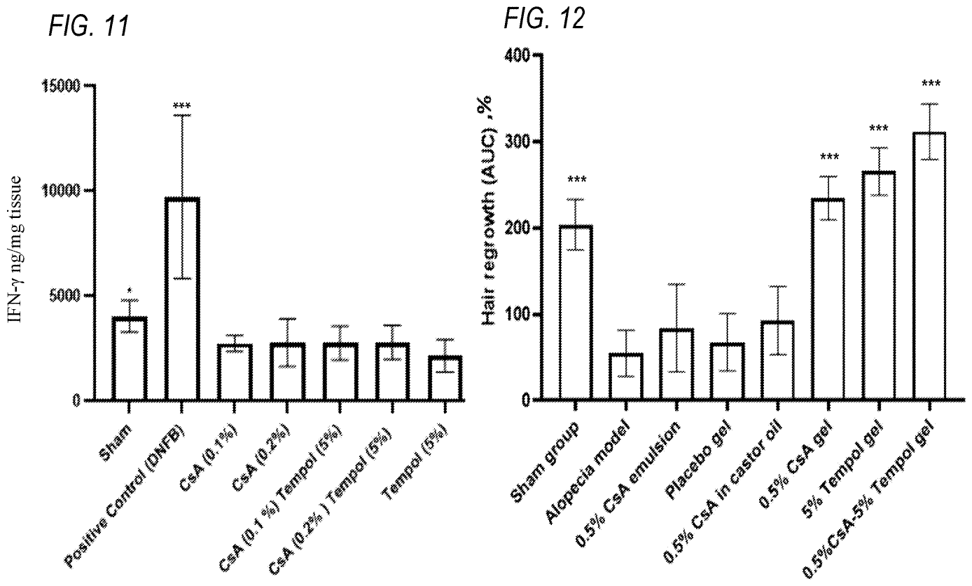

Subsequent analysis of the main proinflammatory cytokines in the ear tissue further supported the histological observations (FIGS. 9-11). Both treatments with CsA and Tempol gel formulations succeeded to restore normal or almost normal levels of TNF-α, IL-6, and to a lesser extent INF-7, at the DNFB-induced site up to the level that was similar to sham.

### Example 4: Enhanced Effect of the Tempol-CsA Gels on Restoration of Hair Growth

### 4.1 Material and Methods

### 4.1.1 In Vivo Alopecia Model

Hair restoration studies used a well-established animal model for AGA. Briefly, mice (8 weeks old female C57BL/6 mice in telogen phase) were subjected to the induction of AGA by intraperitoneal injection of 5α-androstane-3β,17β-diol (DHT, 20 mg/kg), wherein 10 mg 5α-androstane-3α,17β-diol were dispersed in equal volumes of ethanol, 1.2 propanediol and saline solution (1 mL), the injection used DHT dispersion (60 μL) and 2 hydroxypropyl-β-cyclodextrin solution (140 μL). Dorsal coal hair was removed prior to the experiment, all mice presented hairs in telogen phase (no dark skin) prior to treatment. Each group was housed separately and fed pelleted food and water ad libitum.

A preliminary study included: sham mice (a), AGA model (no treatment) (b), and groups treated with 0.5% CsA gel (c) blank gel (d), 0.5% CsA nanoemulsion (e), 0.5% CsA in castor oil (f) 5% Tempol gel (g) or 5% CsA-5% Tempol gel (h) (N=8 per group). Treatments were applied daily (50 mg each formulation) on shaved back area (4 cm²), DHT was injected weekly, once. Every alternate day mice were photographed, and hair growth was quantified by Fiji ImageJ software. After the treatment period of (2 weeks), mice were sacrificed and only those developing AGA (80%) were included in further studies (N=8).

A subsequent study tested treatments with selected gel formulations with clinically relevant concentrations of 0.1% CsA and 0.5% Tempol, the experimental groups included: sham (a), AGA model (no treatment) (b) and groups treated with 0.1% CsA gel (c), 0.5% Tempol gel (d) or 0.1% CsA-0.5% Tempol gel (e) (N=5 per group).

### 4.1.2 Histological Analysis

Dorsal skin and ear samples were fixed in 4% formaldehyde (pH 7.4) overnight and kept in 70% ethanol for a week. Subsequently, samples were embedded in paraffin, sectioned, and stained (H & E, hematoxylin and eosin staining).

### 4.2 Results and Discussion

### 4.2.1 Observations in AGA Model In Vivo

A preliminary experiment tested the formulations (CsA nanoemulsion, CsA in castor oil, CsA gel, Tempol (B gel) and CsA-Tempol gel) with a higher concentration of the CsA active (0.5% CsA). Hair growth was assessed on days 8, 10, 12 and 14 by visual evaluation (not shown). The results suggested that only sham and CsA-Tempol groups had significant hair regrowth on day 12 and full restoration on day 14, while treatments with single actives (CsA or Tempol) showed only a partial hair restoration. No growth restoration was observed in AGA model (no treatment) and treatment with blank gel even on day 14.

Quantitative analysis of hair regrowth (pixel density by AUC %) supported superior performance of the CsA-Tempol gel compared to Tempol and CsA gels and other treatments (FIG. 12). The effect of the 0.5% CsA-5% Tempol gel on hair growth restoration was essentially similar to sham (no statistical differences), when the statistical analyses compared to AGA model. Additional statistical analyses comparing the effect of formulations to the CsA gel suggested that the restorative effect of the CsA/Tempol gel combination can surpass sham and the other Tempol and CsA gels.

### 4.2.2 Histological Analysis of AGA Model In Vivo

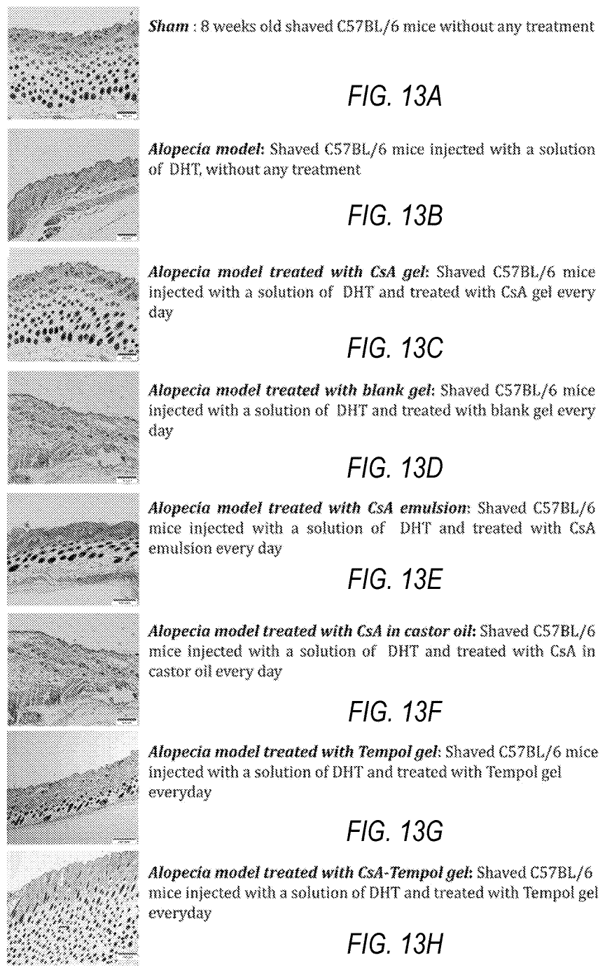

Further histological analysis of skin tissues obtained from the preliminary study supported a strong immunosuppressive effect of the gel formulations (FIG. 13). While sham presented with a thick dermis and numerous hair follicles as opposed to AGA model with a thin dermis and lack of hair follicles, the effect of the 0.5% CsA-5% Tempol gel was the closest to sham in terms of recovery of the primary dermal morphology. The 0.5% CsA gel also showed relatively high degree of dermal preservation. The other treatments, 0.5% CsA nanoemulsion, 0.5% CsA in castor oil and blank gel were essentially ineffective.

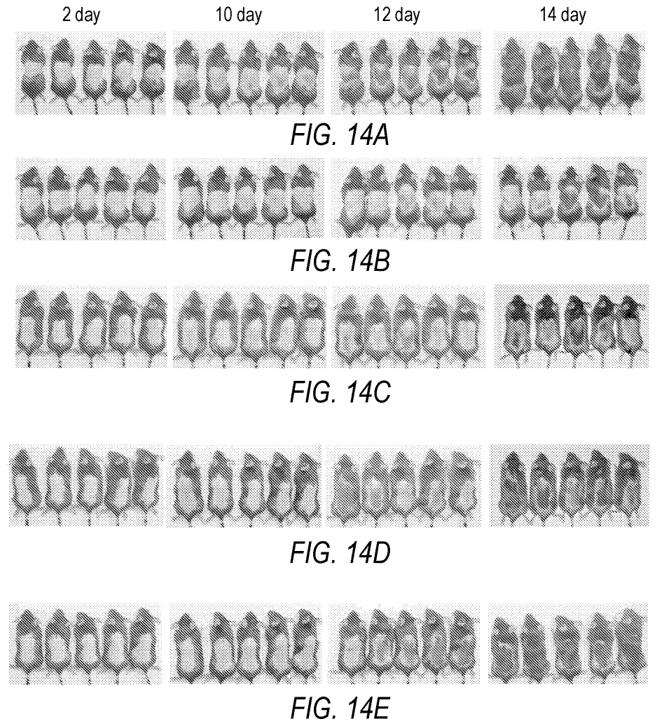

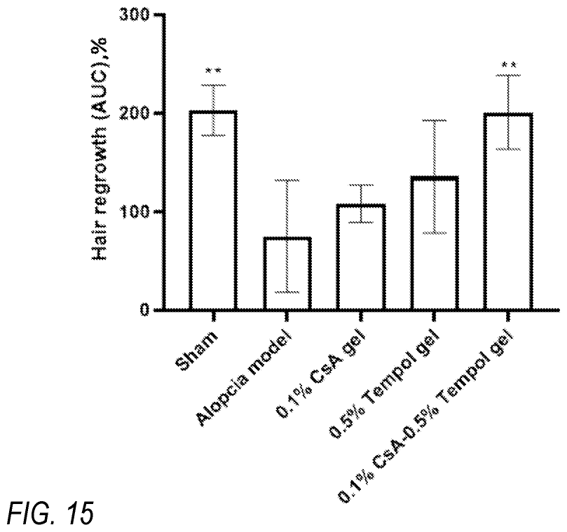

Another study tested formulations with clinically relevant concentrations of 0.1% CsA and 0.5% Tempol (CsA, Tempol and CsA-Tempol gels), the formulations were tested in AGA model. The animals were subjected to visual evaluation on days 2, 10, 12 and 14 (FIG. 14), and quantitative analysis of hair regrowth (FIG. 15). The results of this study reproduced the superior effect of the CsA-Tempol gel formulation even with lower concentrations of actives, suggesting surprisingly enhanced properties of the CsA-Tempol combination in a gel formulation as opposed to gel formulations with single actives.

Overall, the observations in animal model in vivo suggest that the combination of CsA-Tempol in a gel formulation is surprisingly effective and safe for topical treatment alopecia and enhancement of hair growth, in general, and is further effective for reducing local dermal inflammation and reconstitution of primal dermal histomorphology. Moreover, the effects of CsA-Tempol gel formulation proved to be superior, and potentially synergistic, to the effects of the gel formulations with single actives, and sufficiently noticeable in terms of hair regrowth even with relatively low concentrations of 0.1% CsA and 0.5% Tempol. This last point is especially important in view of known adverse reactions that were related to topical and systemic use of CsA.

1.-56. (canceled)

57. A topical formulation comprising a matrix polymeric emulsifier; and a nanoemulsion comprising an immunosuppressant and a cyclic nitroxide.

58. The formulation according to claim 57, wherein the matrix polymeric emulsifier is a high molecular weight copolymer of acrylic acid and an acrylate.

59. The formulation according to claim 57, wherein the formulation comprises at least one thickening agent.

60. The formulation according to claim 57, wherein the nanoemulsion and the matrix polymeric emulsifier are provided as a stable gel.

61. The formulation according to claim 57, wherein the immunosuppressant is selected amongst such agents that reduce or suppress the activity of an in vivo immune system, optionally selected from a steroid agent, a cell proliferation inhibitor, an antibody, an immunophilin-based drug, mycophenolate, and a tumor necrosis factor (TNF-α) inhibitor.

62. The formulation according to claim 61, wherein the immunosuppressant is selected from azathioprine (Imuran), Cyclosporine A (CsA), Mercaptopurine (Purinethol, 6-MP), rapamycin, fujimycin and methotrexate.

63. The formulation according to claim 62, wherein the immunosuppressant is CsA.

64. The formulation according to claim 57, the formulation comprising:
a matrix of a crosslinked copolymer of acrylic acid and a C₁₀-C₃₀ alkyl acrylate; and
a nanoemulsion comprising a lipophilic immunosuppressant, and a cyclic nitroxide, wherein the cyclic nitroxide is a compound of a formula I: 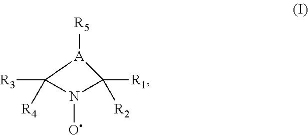
wherein
A represents a carbon atom or a carbon chain comprising up to three carbon atoms, wherein one of the carbon atoms is substituted with an oxygen atom or an oxygen containing group, or wherein one or more of the carbon atoms is substituted with one or two bromine atoms,
each of R₁, R₂, R₃ and R₄, independently, is selected from H and C₁-C₅ alkyl; or
each of R₁ and R₂ together with the carbon atom to which they are attached form a 3- to 7-membered cyclic ring, and/or each of R₃ and R₄ together with the carbon atom to which they are attached form a 3- to 7-membered cyclic ring,
R₅ represents a group selected from aldehydes, ketones, carboxylic acids, carbonyl groups, —O—, —S—, —OH, —SH, —COOH, —COONH₂, —CN, and primary-, secondary-, tertiary- or quaternary-amines, and wherein
O· represents an oxygen radical.

65. The formulation according to claim 64, wherein the cyclic nitroxide compound of formula (I) is a compound of formula (II): 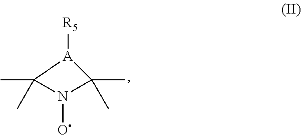
wherein each of A and R₅ is as defined in claim 64.

66. The formulation according to claim 57, wherein the cyclic nitroxide is Tempol:

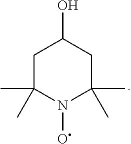

67. The formulation according to claim 57, wherein the cyclic nitroxide compound is of the formula:

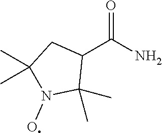

68. The formulation according to claim 57, wherein the cyclic nitroxide compound is of the formula:

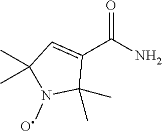

69. The formulation according to claim 57, wherein the cyclic nitroxide is selected from 3-Carbamoyl-PROXYL, 4-hydroxy-2,2,6,6-tetramethylpiperidin-1-oxyl, and 3-Carbamoyl-2,2,5,5-tetramethyl-3-pyrrolin-1-oxyl.

70. The formulation according to claim 57, wherein the nanoemulsion comprises CsA, and a cyclic nitroxide selected from 3-Carbamoyl-PROXYL, 4-hydroxy-2,2,6,6-tetramethylpiperidin-1-oxyl, and 3-Carbamoyl-2,2,5,5-tetramethyl-3-pyrrolin-1-oxyl.

71. The formulation according to claim 57, wherein the formulation comprises at least one of:
a matrix of a crosslinked copolymer of acrylic acid and a C₁₀-C₃₀ alkyl acrylate, and
a nanoemulsion comprising CsA, and a cyclic nitroxide selected from 3-Carbamoyl-PROXYL, 4-hydroxy-2,2,6,6-tetramethylpiperidin-1-oxyl, and 3-Carbamoyl-2,2,5,5-tetramethyl-3-pyrrolin-1-oxyl.

72. The formulation according to claim 57, wherein the nanoemulsion is entrapped in the crosslinked copolymer of acrylic acid and C₁₀-C₃₀ alkyl acrylate matrix to the extent of 60% to 100%.

73. A method for treating male or female alopecia, in a subject in need thereof, the method comprising topical administering to the subject a therapeutically effective amount of the formulation according to claim 57.

74. A method of treating or promoting hair growth and/or reconstitution of dermal histomorphology on the human skin or scalp, the method comprising topical administering to a subject a therapeutically effective amount of the formulation according to claim 57.

75. A method of treating inflammatory, immune and/or auto-immune skin conditions in a subject, the method comprising topical administering to the subject a therapeutically effective amount of the formulation according to claim 57.

76. A formulation comprising a nanoemulsion of CsA and a cyclic nitroxide selected from 3-Carbamoyl-PROXYL, 4-hydroxy-2,2,6,6-tetramethylpiperidin-1-oxyl, and 3-Carbamoyl-2,2,5,5-tetramethyl-3-pyrrolin-1-oxyl; and a copolymer of acrylic acid and a C₁₀-C₃₀ alkyl acrylate, wherein the formulation is configured for application onto a skin region of a subject.
